(19) United States
(12) Patent Application Publication
(10) Pub. No.: US 2011/0038901 A1
(43) Pub. Date: Feb. 17, 2011

(54) Method for Gentle Mechanical Generation of Finely Dispersed Micro-/Nano-Emulsions with Narrow Particle Size Distribution and Device for Carrying Out Said Method

(75) Inventors: Erich J. Windhab, Hemishofen (CH); Verena Schadler, Ede (NL); Beat Troxler, Rain (CH); Andreas Kurt Dürig, Visalia, CA (US); Fred-Rainer Grohmann, Urdorf (CH)

Correspondence Address: ANTONELLI, TERRY, STOUT & KRAUS, LLP, 1300 NORTH SEVENTEENTH STREET, SUITE 1800, ARLINGTON, VA, 22209-3873, US

(73) Assignees: ETH-ZURICH, INSTITUT FUR LEBENSMITTELWISSENSCHAFT,, Zurich (CH); KINEMATICA AG, Littau (CH); ION BOND AG, Olten (CH); PROCESSTECH GMBH, Hindelbank (CH)

(21) Appl. No.: 11/574,152

(22) PCT Filed: Aug. 19, 2005
(86) PCT No.: PCT/EP05/08980

§ 371 Date: Dec. 27, 2007

(30) Foreign Application Priority Data
Aug. 23, 2004 (DE) 10 2004 040 735.5

(51) Int. Cl. B01F 3/08; A23P 1/00; B01F 5/06; A61K 8/06; A61K 8/02; A61K 9/107

(52) U.S. Cl. 424/401; 426/519; 366/176.2; 512/4; 424/486

(57) ABSTRACT

This invention relates to a method for the mechanically protective production of finely dispersed micro-/nanoemulsions with narrow droplet size distribution, whereby drops are produced on the surface of a membrane or of a filter fabric, and the drops are detached from the membrane or filter fabric surface by motion of the membrane or of the filter fabric in a first immiscible liquid phase in which pronounced stretching flow components in particular, besides shear flow components, bring about the detachment of the drops formed on the membrane surface especially efficiently and protectively. The invention also relates to a device for implementing the method according to the invention with a membrane or filter unit that is positioned to move, in particular to be able to rotate, in a housing with a gap that may be eccentric toward the inner wall of the housing and/or provided with flow baffles that produce stretching flow components.

This invention relates to a method for mechanically protective production of finely dispersed micro-/nanoemulsions with a narrow droplet size distribution.

The invention also relates to a device for implementing the method.

### PRIOR ART

The preparation of finely dispersed emulsions is an important development objective for the food, pharmaceutical, cosmetics, and chemical industries. The reason for this is the ability to keep such emulsions stable against settling with sufficiently small dispersed droplets, and to utilize the extremely large internal interface for the adsorption of functional ingredients (for example drugs, perfumes, pigments, etc.). The dispersed droplets also permit the buildup of particle networks that selectively influence the rheological properties of such emulsions.

Membrane emulsification methods are a new field for the manufacturers of machines and apparatus. Rotor/stator dispersing systems and high-pressure homogenization are ordinarily used for fine emulsification. Droplet dispersion in these apparatuses occurs under extremely high mechanical stress on both the dispersed and continuous phases. The membrane emulsification methods that have existed for about five years are very protective from the mechanical viewpoint compared to the conventional methods mentioned above, since the finely dispersed emulsion droplets are not produced by breaking apart larger drops, but are formed and released in their final size at the discharge orifices of the membrane pores.

In continuous membrane processes existing up to now, the continuous emulsion liquid phase flows tangentially over the membrane in the form of a pure shear flow. The shear stresses acting on the drops and detaching them from the membrane are not very efficient or not at all efficient with regard to detaching small drops and further dispersing (splitting) them, especially in case of high drop viscosities. This represents a considerable drawback with regard to the ability to adjust for small drop sizes and narrow droplet size distributions with the output capacities generally prescribed within narrow limits in the industrial production of emulsion systems.

### Task

The task underlying this invention is to provide a method for the mechanically protective production of finely dispersed micro-/nanoemulsions with narrow droplet size distribution.

The task underlying the invention is also to make available a device for implementing the method according to the invention.

### Accomplishment of the Task Related to the Method

This task is accomplished by the features described in Claim 1.

### Some Advantages

A stretching flow component superimposed on a tangential shear flow on the rotating membrane surface in the method according to the invention makes possible the protective detachment of smaller droplets, and their more efficient further dispersion after detachment takes place than is the case with pure shear flows.

In the method according to the invention, emulsion drops are produced on the surface of a membrane or a filter fabric permeated with pores, by a first fluid phase being pressed through these pores and by the drops being stripped from the membrane surface by its rotational motion in a second liquid phase immiscible with the first. Detachment of the liquid drops from the membrane surface is brought about by tangential and perpendicular stresses acting on them caused by the flow, assisted by additional centrifugal forces. The preferred use of membranes with definite large pore separations (≧2 x) compared to the pore diameter x is also necessary for producing a narrow droplet size distribution in the emulsion generated. The tangential flow over the membrane accomplished according to the invention with additionally efficient stretching flow components permits the production of distinctly smaller droplet diameters than conventional membrane emulsification methods with fixed or rotating membranes with pure shear flow over them, with comparable pore diameters. Compared to conventional emulsification methods by means of high-pressure homogenizers or rotating rotor/stator dispersing systems, producing emulsion droplets according to the invention offers the advantage of distinctly reduced mechanical stress for comparable diameters of the drops generated. This has advantages with respect to maintaining natural properties of functional components, for example of proteins in the drops or on their interfaces.

### OTHER INVENTIVE EMBODIMENTS

Other inventive embodiments are described in Claims 2 to 10.

### Accomplishment of the Task Related to the Device

This task is accomplished by the features described in Claim 11.

### Some Advantages

The device according to the invention permits simple modification and adaptation of the stretching flow-tangential flow characteristic of the membrane with respect to the fraction of stretching flow in the total flow, by varying the eccentricity of the rotating membrane cylinder and/or easily interchangeable flow baffles.

The device according to the invention is of very compact construction since the membrane unit can be placed in the housing closely spaced from its inner wall.

### OTHER INVENTIVE EMBODIMENTS

These are described in Claims 12 to 26.

Other features and advantages are found in the following description of the drawings in which the invention is illustrated by way of example. The drawings show:

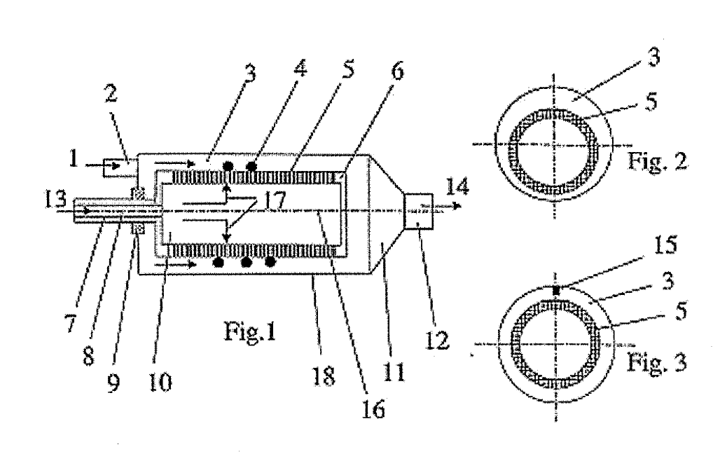

FIG. 1 A device according to the invention in longitudinal axial cross section, wherein the cut walls are not hatched, for simplification;

FIG. 2 a cross section of the device shown in FIG. 1 orthogonal to the longitudinal axis;

FIG. 3 likewise, a cross section of a device according to the invention orthogonal to the longitudinal axis, in another embodiment with flow baffles;

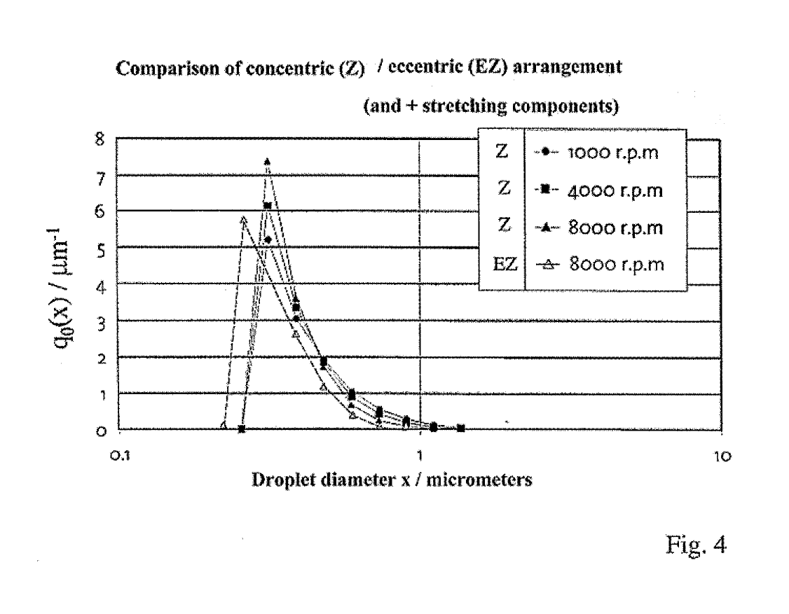

FIG. 4 a graphic illustration of the number density droplet distribution (q₀ distribution) that was recorded for water droplets in sunflower oil with filter unit or membrane unit at speeds of 1000 to 8000 rpm; and

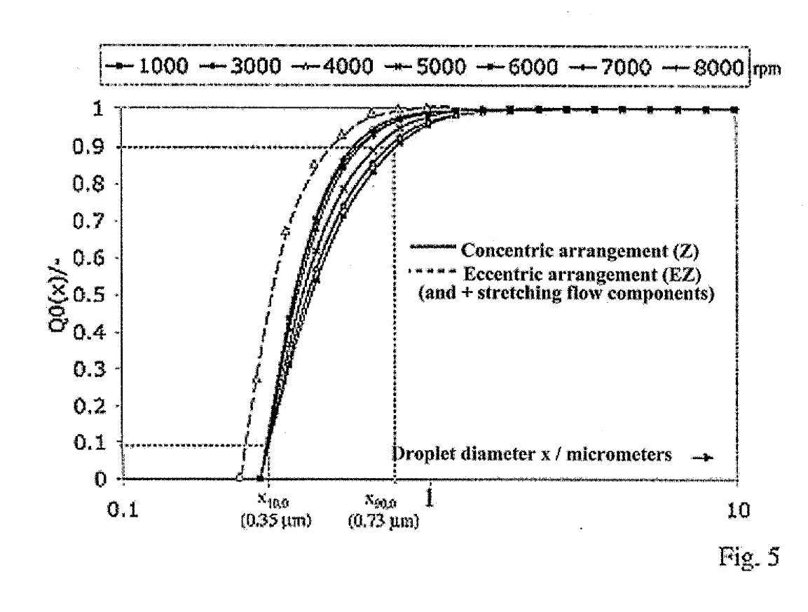

FIG. 5 a graphic illustration of the total number droplet distribution (Q₀ distribution) that was recorded for water droplets in sunflower oil with filter unit or membrane unit at speeds of 1000 to 8000 rpm (so-called Q₀(x) distributions), plotting the characteristic droplet sizes X_{90.0} and x_{10.0}, the ratio of which ((x_{90.0}/x_{10.0}) is used as a suitable measure of the spread of droplet size distribution, for concentric arrangement (Z) and eccentric arrangement (EZ).

Reference symbol 1 designates a continuous liquid phase that is fed by pump from a suitable supply reservoir (not shown) to a connector 2 and through this to a gap 3.

Dispersed drops are labeled 4, and a membrane unit or filter fabric unit is labeled 5, while 6 identifies a cylindrical body made as a membrane cylinder.

7 is a rotating hollow shaft that has a bore 8 in its center. The shaft 7 is sealed off by a dynamic rotating mechanical seal 9.

The bore 8 opens into an internal space 10 in the filter fabric unit or the membrane unit 5.

A conical component is positioned at 11 that exits into an outflow port 12. The conical component 11 and the outflow port 12 constitute part of a housing 18.

A dispersion liquid phase is fed in at 13 by a motorized pump from a container, also not shown.

The emulsion 14 leaves the housing 18 through the outflow port 12.

In the embodiment shown in FIGS. 1 and 2, the filter fabric unit or membrane unit 5 is arranged eccentrically relative to the housing 18, with definite adjustable eccentricity.

In the embodiment according to FIG. 3, there is a flow baffle (for example the ridge 15) in the gap 3, which extends along the longitudinal axis 15 of the housing 18. The ridge 15 can also run helically, or can be part of a spiral. It is also possible to provide a number of such ridges 15, spirals, or helical ridges 3 with different cross sectional geometries inside the gap 3.

The diametrically opposite-pointing arrows 17 are intended to indicate the approximately radially oriented direction of flow of the dispersed liquid phase 13 with respect to the filter fabric unit or the membrane unit 5.

FIG. 5 illustrates a corresponding total count distribution Q₀(x) plotting the characteristic droplet sizes x_{90.0} and x_{10.0}, the ratio of which (x_{90.0}/x_{10.0}) is used as a suitable measure of the breadth of droplet size distribution, showing representations for concentric positioning (Z) and eccentric positioning (EZ) (and/or with stretching flow components).

The way the embodiment shown in the drawing operates is as follows:

The dispersion liquid phase 13 is forced by the motor-driven pump, not shown, through the rotating hollow shaft 7 with an internal bore 8 into the interior chamber 10 of the rotating membrane cylinder unit 6. The shaft 7 is sealed off from the housing 18 by means of the rotating mechanical seal 9. From there, the dispersion liquid phase 13 passes through the membrane 5 attached on the surface of the cylinder body and forms the dispersed drops 4 on its outside.

The continuous liquid phase 1 is introduced through the connector 2 into the cylindrical housing 18, and flows axially through the gap 3 between the rotating membrane unit or filter fabric unit 5 and the housing 18. It impinges on the dispersed drops 4 formed on the membrane surface. The intensity of the impinging flow is determined by the circumferential velocity of the membrane unit or filter fabric unit and cylinder 6, the gap width 3, and the eccentricity, and flow baffles (such as ridge(s), pins, knives/scrapers) fastened to the outer cylinder wall between it and the housing 18.

If there is an eccentric positioning of the membrane cylinder 6 in the cylindrical housing 18 (FIG. 2) between the membrane cylinder 6 and the housing 18, a mixed shear/stretching flow occurs that has improved dispersing power. To produce improved drop detachment from the membrane surface, the flow baffles (e.g., ridge 15) that interfere specifically with the rotational flow can also be attached, preferably on the inner wall of the housing according to the invention. Such flow baffles (e.g., ridge 15) can be fitted either in a straight line with axial orientation, or helically.

The mixture of dispersed drops 4 and continuous liquid phase 1, the emulsion 14, is formed at the outlet from the gap 3 in an outlet geometry that preferably consists of a conical component 11 and an outlet port 12.

In FIG. 4, emulsions produced by means of a rotating membrane (CPDN membrane Controlled Pore Distance Membrane) are illustrated graphically as a droplet size distribution function (number distribution qo(x)) in a comparison of pure shear flow (concentric cylinder) and superimposed stretching flow (eccentric cylinder).

The features described in the Abstract, in the Claims, and in the Specification, as well as features apparent from the drawing, may be important both individually and in any combination for realization of the invention.

### LIST OF REFERENCE SYMBOLS

1 Liquid phase, continuous

2 Connector, connecting ports

3 Gap, annular gap, gap width

4 Drops, dispersed

5 Membrane, membrane unit, filter fabric unit

6 Cylinder body, membrane cylinder

7 Rotating shaft, shaft, hollow shaft

8 Bore, internal

9 Rotating mechanical seal, dynamic

10 Internal chamber

11 Component, conical

12 Outlet port

13 Liquid phase, dispersed

14 Emulsion

15 Ridge

16 Longitudinal axis

17 Double arrow

18 Housing

### LITERATURE REFERENCES

DE 101 27 075 C2

WO 2004/030799 A1

WO 01/45830 A1

U.S. Pat. No. 5,326,484

1. Method for the mechanically protective production of finely dispersed micro-/nanoemulsions with narrow droplet size distribution, whereby drops (4) are produced by a filter fabric unit or a membrane unit (5) with pores in which a first liquid phase (13) moves through these pores, and in particular is forced through them, and the drops (4) are moved away (detached) from the filter fabric or membrane surface by their inherent motion in a second liquid phase immiscible with the first liquid phase (1) while superimposed shear flow components and pronounced stretching flow components are produced in the gap between the membrane cylinder and the wall of the housing.

2. Method according to claim 1, characterized in that the filter fabric unit or the membrane unit (5) is rotated at an adjustable constant speed.

3. Method according to claim 1, characterized in that the filter fabric unit or the membrane unit (5) is rotated at a periodically oscillating speed.

4. Method according to claim 1 or one of the succeeding claims, characterized in that the dispersed liquid phase (13) flows through the filter fabric or membrane unit (5) continuously or in pulses.

5. Method according to claim 1 or one of the succeeding claims, characterized in that before the dispersed liquid phase (13) flows through the filter fabric or membrane unit (5), the continuous liquid phase (1) or another liquid immiscible with the dispersed liquid phase (13) briefly flows through the filter fabric or membrane pore system in order to wet the filter fabric or membrane pore walls of the filter fabric or membrane unit (5) to make them repellent to the dispersed liquid phase (13).

6. Method according to claim 1, characterized in that the filter fabric or membrane unit (5) is rotated at a speed that is not periodically variable—chiefly according to a program stored in a computer.

7. Method according to claim 1 or 2, characterized in that the motion of the filter fabric or membrane unit (5) sets predetermined definite shear stresses and/or stretching stresses on the emulsion drops (4) formed on the filter fabric or membrane surface.

8. Method according to claim 1, characterized in that the filter fabric or membrane unit experiences additional flow perpendicular to the circumferential direction of rotation, for example in the radial direction in the case of a disk-shaped filter fabric or membrane unit, or in the axial direction in the case of a cylindrical filter fabric or membrane unit (5).

9. Method according to claim 1, characterized in that the liquid phase (13) flowing through the filter fabric or membrane unit (5) for its part represents an emulsion, and thus a double emulsion of the water/oil/water or oil/water/oil type is formed in another liquid phase after the drops (4) depart from the filter fabric or membrane surface.

10. Method according to claim 1, characterized in that the liquid phase (1) that is fed past the surface of the filter fabric or membrane unit (5) for its part represents a suspension, which forms a suspension/emulsion system in another surrounding liquid phase after detachment of the drops (4).

11. Device for implementing the method of claim 1 or one of the succeeding claims, with a preferably rotationally symmetrical filter fabric and membrane unit (5) movable around its longitudinal axis by a motor, which is positioned in a housing (18) with a surrounding gap (3) of variable gap width.

12. Device according to claim 11, characterized in that the filter fabric or membrane unit (5) is in the shape of a cylinder.

13. Device according to claim 11, characterized in that the filter fabric or membrane unit (5) is in the shape of a disk.

14. Device according to claim 11, characterized in that the inner wall of the housing (18) bounding the gap (3) and the filter fabric or membrane unit (5) are arranged eccentrically with respect to one another.

15. Device according to claim 11, characterized in that there is/are one or more ridge(s) (15) in the gap (3) as generator(s) for stretching flow components.

16. Device according to claim 15, characterized in that the ridge (15) involved extends in the longitudinal direction of the housing (18) and of the filter fabric and membrane unit 5.

17. Device according to claim 15 or 16, characterized in that the ridge (15) involved is made in straight or helical form, or as a screw-like spiral.

18. Device according to claim 15 or one of the succeeding claims, characterized in that the ridge (15) involved is positioned on the inner wall of the housing (18).

19. Device according to claim 11 or one of the succeeding claims, characterized in that the circumferential velocity of the rotating driven filter fabric or membrane unit (5) is between 1 m/s and 50 m/s.

20. Device according to claim 11 or one of the succeeding claims, characterized in that the axial velocity of the flow of the continuous liquid phase (1) over the cylindrical filter fabric or membrane unit (5) can be set independently of the circumferential velocity of the filter fabric or membrane unit (5), and in particular it is controllable or can be regulated.

21. Device according to claim 11 or one of the succeeding claims, characterized in that the dispersed liquid phase (13) is fed through a hollow shaft (7) to the filter fabric or membrane unit (5) connected to it, and can be forced through this by pump pressure, so that dispersed liquid drops (4) can be produced on the surface of the filter fabric or membrane unit.

22. Device according to claim 11 or one of the succeeding claims, characterized in that the inherent motion of the filter fabric or membrane unit (4) is adjustable with a control device or regulator.

23. Device according to claim 11 or one of the succeeding claims, characterized in that the inherent motion of the filter fabric or membrane unit (5) can be executed through a computer program.

24. Device according to claim 11 or one of the succeeding claims, characterized in that the inherent motion of the filter fabric or membrane unit (5) can be reversed after a predetermined period of time.

25. Device according to claim 11 or one of the succeeding claims, characterized in that the motorized drive for the pump transporting the liquid phase (13) can be driven intermittently (pulsed) according to a predetermined program.

26. Device according to claim 11 or one of the succeeding claims, characterized in that the motorized drive for the pump transporting the dispersed phase (1) can be driven intermittently (pulsed) according to a predetermined program.
